(19) United States
(12) Patent Application Publication
(10) Pub. No.: US 2008/0097139 A1
(43) Pub. Date: Apr. 24, 2008

(54) SYSTEMS AND METHODS FOR TREATING LUNG TISSUE

(75) Inventors: Claude Clerc, Marlborough, MA (US); Paul M. Scopton, Winchester, MA (US); Isaac Ostrovsky, Wellesley, MA (US); Harold M. Martins, Newton, MA (US); Stephen J. Perry, Shirley, MA (US)

Correspondence Address: VISTA IP LAW GROUP LLP, 12930 Saratoga Avenue, Suite D-2, Saratoga, CA, 95070, US

(73) Assignee: Boston Scientific Scimed, Inc., Maple Grove, MN (US)

(21) Appl. No.: 11/457,646

(22) Filed: Jul. 14, 2006

(51) Int. Cl. A61M 36/12; A61B 18/00; A61B 18/12

(52) U.S. Cl. 600/7; 606/41; 604/506; 601/2; 606/20; 606/27

(57) ABSTRACT

A system for treating lung tissue includes a tube having a distal end, an anchoring device secured to the tube, the anchoring device configured to anchor at least a portion of the tube against an esophagus, a trachea, or a bronchus; and an ablation device carried within a lumen of the tube.

### FIELD OF INVENTION

This application pertains to systems and methods for treating lung tissue, and more specifically, to systems and methods for performing lung volume reduction.

### BACKGROUND

Emphysema is a medical condition characterized by breakdown of surfactant and elastic proteins at the alveolar level, which leads to hyper-inflated lung regions. The hyper-inflated lung regions interfere with expansion, contraction, and gas exchange in the remaining healthy lung tissue.

In a lung volume reduction procedure, one or more hyper-inflated lung regions are resected and removed from the patient, thereby preventing the hyper-inflated lung regions from interfering with the function of the remaining healthy lung tissue. Such procedure poses many risks. For example, significant bleeding at the treated regions may result from such procedure. In some cases, a patient may even die from such bleeding. In addition, after a portion of a lung has been cut away, the surface at the cutting plane of the remaining lung will need to be sealed to thereby prevent gas (e.g., inhaled gas) from escaping from the lung into the patient's body.

### SUMMARY

In accordance with some embodiments, a method of treating lung tissue includes (i) inserting a distal end of a cannula into a trachea, the cannula further having a proximal end and a lumen extending between the distal and proximal ends; (ii) placing the cannula distal end into a bronchus; (iii) deploying an ablation device from the distal end of the cannula; and (iv) using the ablation device to ablate at least a portion of the lung tissue to thereby reduce a volume of the portion of the lung tissue. In various embodiments, the ablation device may comprise an electrode, a needle configured for delivering fluid, an ultrasound transducer, a radiation seed, or a cryogenic device. In some embodiments, the method may further comprise delivering a fluid into the bronchus. By way of non-limiting examples, the fluid may be electrically conductive fluid, a may contain toxic agent. The fluid temperature may be hot (e.g., above 50° C.) or cooled (e.g., below 3° C.).

In accordance with further embodiments, a method of treating lung tissue includes (i) creating an opening through a patient's skin such that a surface of a portion of a lung can be viewed through the opening; (ii) deploying an ablation device into lung tissue; and (iii) using the ablation device to ablate at least a portion of the lung tissue to thereby reduce a volume of the portion of the lung tissue. The ablation energy is preferably delivered by the ablation device to ablate the at least a portion of the lung tissue until the surface subsides. In various embodiments, the ablation device may comprise an electrode, a needle configured for delivering fluid, an ultrasound transducer, a radiation seed, or a cryogenic device. In some embodiments, the method may further comprise delivering a fluid into at least a portion of the lung. By way of non-limiting examples, the fluid may be electrically conductive fluid, a may contain toxic agent. The fluid temperature may be hot (e.g., above 50° C.) or cooled (e.g., below 3° C.).

In accordance with yet other embodiments of the invention, a system for treating lung tissue includes a fluid delivery tube having a proximal end, a distal end, and a lumen extending between the proximal and distal ends, the fluid delivery tube having a cross-sectional dimension sized for insertion into a trachea, and a container coupled to the proximal end of the fluid delivery tube, the container in fluid communication with the fluid delivery tube, and carrying fluid for treating lung tissue, wherein the fluid, when delivered within a lung, causes a volume of the lung tissue to reduce.

In accordance with yet further other embodiments, a system for treating lung tissue includes a tube having a distal end, an anchoring device secured to the tube, the anchoring device configured to anchor at least a portion of the tube against an esophagus, a trachea, or a bronchus, and an electrode located within a lumen of the tube.

Other and further aspects, features and embodiments will be evident from reading the following detailed description of the drawings.

### BRIEF DESCRIPTION OF THE DRAWINGS

Embodiments of the invention are illustrated by way of example, and not by way of limitation, in the figures of the accompanying drawings, in which like reference numerals refer to like components, and in which:

FIG. 1 illustrates a lung treatment system having a treatment assembly in accordance with some embodiments;

FIG. 2 illustrates the treatment assembly of FIG. 1, showing the treatment assembly having retracted electrodes;

FIG. 3 illustrates the treatment assembly of FIG. 1, showing the treatment assembly having deployed electrodes;

FIGS. 4A-4C illustrate a method of using the lung treatment system of FIG. 1 in accordance with some embodiments;

FIG. 5 illustrates a lung treatment system in accordance with other embodiments; and

FIG. 6 illustrates a lung treatment system in accordance with other embodiments.

### DESCRIPTION OF THE ILLUSTRATED EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of specific embodiments. They are not an exhaustive description of the invention or as a limitation on the scope of the invention. In addition, each illustrated embodiment may not incorporate all the aspects or features, and an aspect or an advantage described in conjunction with a particular embodiment is not necessarily limited to that embodiment, but can be included in any of a number of other embodiments, even if not so illustrated.

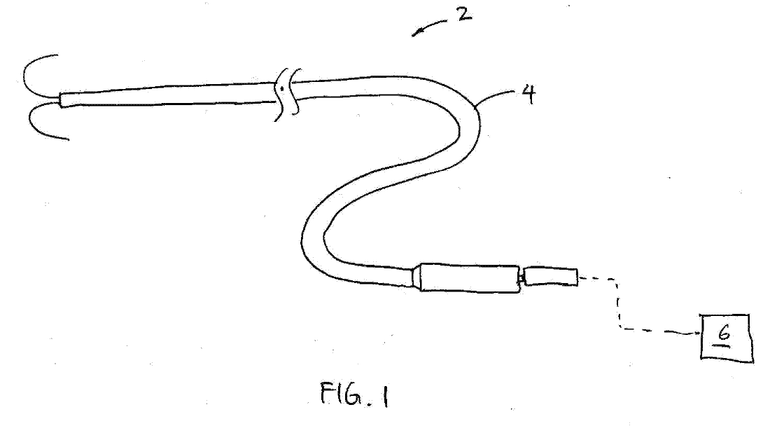

FIG. 1 illustrates a system 2 for treating lung tissue in accordance with some embodiments of the invention. The system 2 includes a treatment assembly 4 configured for introduction into the body of a patient for ablative treatment of target tissue, and a generator 6 configured for supplying energy to the treatment assembly 4 in a controlled manner.

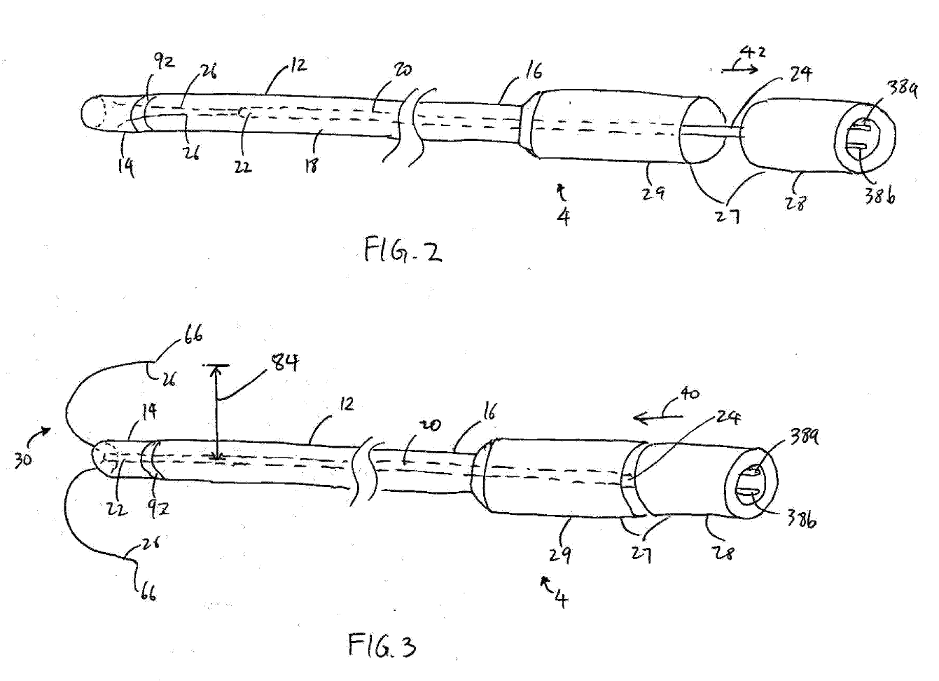

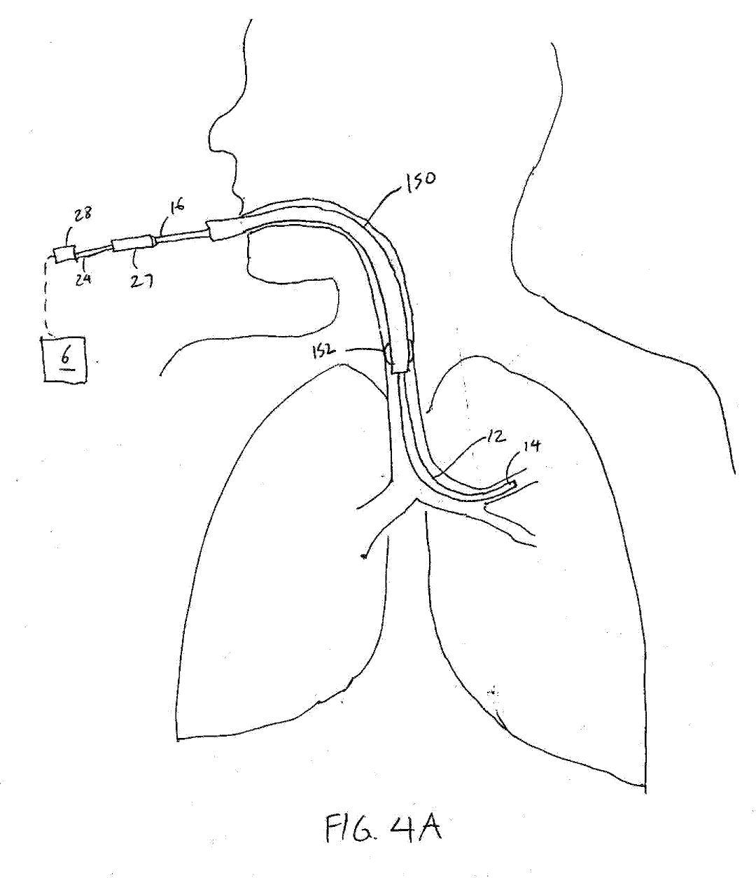

Referring to FIGS. 2 and 3, the treatment assembly 4 includes an elongate cannula 12, a shaft 20 slidably disposed within the cannula 12, and an array 30 of electrodes 26 carried by the shaft 20. The cannula 12 has a distal end 14, a proximal end 16, and a central lumen 18 extending through the cannula 12 between the distal end 14 and the proximal end 16. The cannula 12 may be rigid, semi-rigid, or flexible depending upon the designed means for introducing the cannula 12 to the target tissue. The cannula 12 is composed of a suitable material, such as plastic, metal or the like, and has a suitable length, typically in the range from 5 cm to 150 cm. For example, if the cannula 12 is used endoscopically, then it preferably has a length that is between 50 cm and 150 cm (e.g., around 100 cm). The length of the cannula 12 can also have other dimensions. If composed of an electrically conductive material, the cannula 12 is preferably covered with an insulative material. The cannula 12 has an outside cross sectional dimension sized for insertion into a patient's trachea. The cannula 12 can also have other outside cross sectional dimensions in other embodiments.

It can be appreciated that longitudinal translation of the shaft 20 relative to the cannula 12 in a proximal direction 42 retracts the electrode tines 26 into the distal end 14 of the cannula 12 (FIG. 3), and longitudinal translation of the shaft 20 relative to the cannula 12 in a distal direction 40 deploys the electrode tines 26 out of the distal end 14 of the cannula 12 (FIG. 2). The shaft 20 comprises a distal end 22 and a proximal end 24. Like the cannula 12, the shaft 20 is composed of a suitable material, such as plastic, metal or the like. In the illustrated embodiment, each electrode 26 takes the form of an electrode tine, which resembles the shape of a needle or wire. Each of the electrodes 26 is in the form of a small diameter metal element, which can penetrate into tissue as it is advanced from a target site within the target region.

In some embodiments, distal ends 66 of the electrodes 26 may be honed or sharpened to facilitate their ability to penetrate tissue. The distal ends 66 of these electrodes 26 may be hardened using conventional heat treatment or other metallurgical processes. They may be partially covered with insulation, although they will be at least partially free from insulation over their distal portions.

When deployed from the cannula 12, the array 30 of electrodes 26 has a deployed configuration that defines a volume having a periphery with a radius 84 in the range from 0.5 to 4 cm. However, in other embodiments, the maximum radius can be other values. The electrodes 26 are resilient and pre-shaped to assume a desired configuration when advanced into tissue. In the illustrated embodiments, the electrodes 26 diverge radially outwardly from the cannula 12 in a uniform pattern, i.e., with the spacing between adjacent electrodes 26 diverging in a substantially uniform and/or symmetric pattern.

It should be noted that although a total of two electrodes 26 are illustrated in FIG. 3, in other embodiments, the treatment assembly 4 can have more or fewer than two electrodes 26. In exemplary embodiments, pairs of adjacent electrodes 26 can be spaced from each other in similar or identical, repeated patterns and can be symmetrically positioned about an axis of the shaft 20. It will be appreciated that a wide variety of particular patterns can be provided to uniformly cover the region to be treated. In other embodiments, the electrodes 26 may be spaced from each other in a non-uniform pattern.

The electrodes 26 can be made from a variety of electrically conductive elastic materials. Very desirable materials of construction, from a mechanical point of view, are materials which maintain their shape despite being subjected to high deformation. Certain “super-elastic alloys” include nickel/titanium alloys, copper/zinc alloys, or nickel/aluminum alloys. Alloys that may be used are also described in U.S. Pat. Nos. 3,174,851, 3,351,463, and 3,753,700, the disclosures of which are hereby expressly incorporated by reference. The electrodes 26 may also be made from any of a wide variety of stainless steels or cobalt-base alloy, such as Elgiloy or MP35N. The electrodes 26 may also include the Platinum Group metals, especially platinum, rhodium, palladium, rhenium, as well as tungsten, gold, silver, tantalum, and alloys of these metals. These metals are largely biologically inert, and have significant radiopacity to allow the electrodes 26 to be visualized in-situ, and their alloys may be tailored to accomplish an appropriate blend of flexibility and stiffness. They may be coated onto the electrodes 26 or be mixed with another material used for construction of the electrodes 26.

In the illustrated embodiments, the treatment assembly 4 further includes an electrode 92 secured to the cannula 12. The electrode 92 is operative in conjunction with the array 30 to deliver energy to tissue. The electrodes 26 in the array 30 are positive (or active) electrodes while the operative electrode 92 is a negative (or return) electrode for completing energy path(s). In such cases, energy is directed from the electrodes 26 in the array 30 radially inward towards the electrode 92. Alternatively, the electrode 92 can be active electrode while the electrodes 26 in the array 30 are return electrodes for completing energy path(s), in which cases, energy is directed from the electrode 92 radially outward towards the electrodes 26.

In the illustrated embodiments, the operative electrode 92 has a tubular shape, but can have other shapes in alternative embodiments. In other embodiments, the operative electrode 92 may have a sharp distal tip (not shown) for piercing tissue. In such cases, the operative electrode 92 may be secured to the distal end 14 of the cannula 12 such that the distal tip of the operative electrode 92 is distal to the distal end 14.

In the illustrated embodiments, the array 30 of electrodes 26 and the operative electrode 92 are used to deliver radiofrequency (RF) current in a bipolar fashion, which means that current will pass between the array 30 of electrodes 26 and the operative electrode 92. In a bipolar arrangement, the array 30 and the electrode 92 will be insulated from each other in any region(s) where they would or could be in contact with each other during a power delivery phase. If the cannula 12 is made from an electrically conductive material, an insulator (not shown) can be provided to electrically insulate the operative electrode 92 from the electrodes 26 in the array 30.

In other embodiments, the electrode array 30 can be electrically insulated from the operative electrode 92 by an insulator having other shapes or configurations that is placed at different locations in the treatment assembly 4. For example, in other embodiments, the treatment assembly 4 can include insulators within the respective openings 80. Alternatively, if the cannula 12 is made from a non-conductive material, the insulator is not needed, and the ablation probe 4 does not include the insulator.

Alternatively, the RF current is delivered to the electrode array 30 in a monopolar fashion, which means that current will pass from the electrode array 30, which is configured to concentrate the energy flux in order to have an injurious effect on the surrounding tissue, to a dispersive electrode (not shown), which is located remotely from the electrode array 30 and has a sufficiently large area (typically 130 cm2 for an adult), so that the current density is low and non-injurious to surrounding tissue. In such cases, the electrode assembly 4 does not include the operative electrode 92. The dispersive electrode may be attached externally to the patient, e.g., using a contact pad placed on the patient's flank. In other embodiments, the electrode assembly 4 can include the operative electrode 92 for delivering ablation energy in a monopolar configuration. In such cases, the array 30 of electrodes 26 and the operative electrode 92 are monopolar electrodes, and current will pass from the electrodes 26 and the electrode 92 to the dispersive electrode to thereby deliver ablation energy in a monopolar configuration.

Returning to FIGS. 2 and 3, the treatment assembly 4 further includes a handle assembly 27, which includes a handle portion 28 mounted to the proximal end 24 of the shaft 20, and a handle body 29 mounted to the proximal end 16 of the cannula 12. The handle portion 28 is slidably engaged with the handle body 29 (and the cannula 20). The handle portion 28 also includes two electrical connectors 38a, 38b, which allows the treatment assembly 4 to be connected to the generator 6 during use. Particularly, the electrical connector 38a is electrically coupled to the electrodes 26, and the electrical connector 38b is electrically coupled to the electrode 92. The electrical connector 38a can be conveniently coupled to the electrodes 26 via the shaft 20 (which will be electrically conductive), although in other embodiments, the connector 38a can be coupled to the electrodes 26 via separate wires (not shown). The handle portion 28 and the handle body 29 can be composed of any suitable rigid material, such as, e.g., metal, plastic, or the like. In other embodiments, if the electrode assembly 4 does not include the electrode 92, then the electrode assembly 4 does not include the connector 38b.

In some embodiments, the cannula 12 can include a steering mechanism (not shown) for allowing the distal end 14 of the cannula 12 to be steered during use. For example, in some embodiments, the cannula 12 can include one or more steering wires secured to the distal end 14. During use, tension can be applied to the steering wire(s) to thereby bend the distal end 14 in one or more directions. Alternatively, or additionally, the shaft 20 can also include a steering mechanism. For example, the shaft 20 can include one or more steering wires secured to the distal end 22. During use, tension can be applied to the steering wire(s) to thereby bend the distal end 22 in one or more directions. Steering devices have been described in U.S. Pat. Nos. 5,254,088, 5,336,182, 5,358,478, 5,364,351, 5,395,327, 5,456,664, 5,531,686, 6,033,378, and 6,485,455, the entire disclosures of which are expressly incorporated by reference herein.

Referring back to FIG. 1, the generator 6 is electrically connected to the electrical connectors 38a 38b, which may be directly or indirectly (e.g., via a conductor) electrically coupled to the electrode array 30. The generator 6 is a conventional RF power supply that operates at a frequency in the range from 200 KHz to 1.25 MHz, with a conventional sinusoidal or non-sinusoidal wave form. Such power supplies are available from many commercial suppliers, such as Valleylab, Aspen, and Bovie. More suitable power supplies will be capable of supplying an ablation current at a relatively low voltage, typically below 150V (peak-to-peak), usually being from 50V to 100V. The power will usually be from 20 W to 200 W, usually having a sine wave form, although other wave forms would also be acceptable. Power supplies capable of operating within these ranges are available from commercial vendors, such as Boston Scientific Corporation of San Jose, Calif., which markets these power supplies under the trademarks RF2000 (100 W) and RF3000 (200 W). Other types of power supply may also be used in other embodiments.

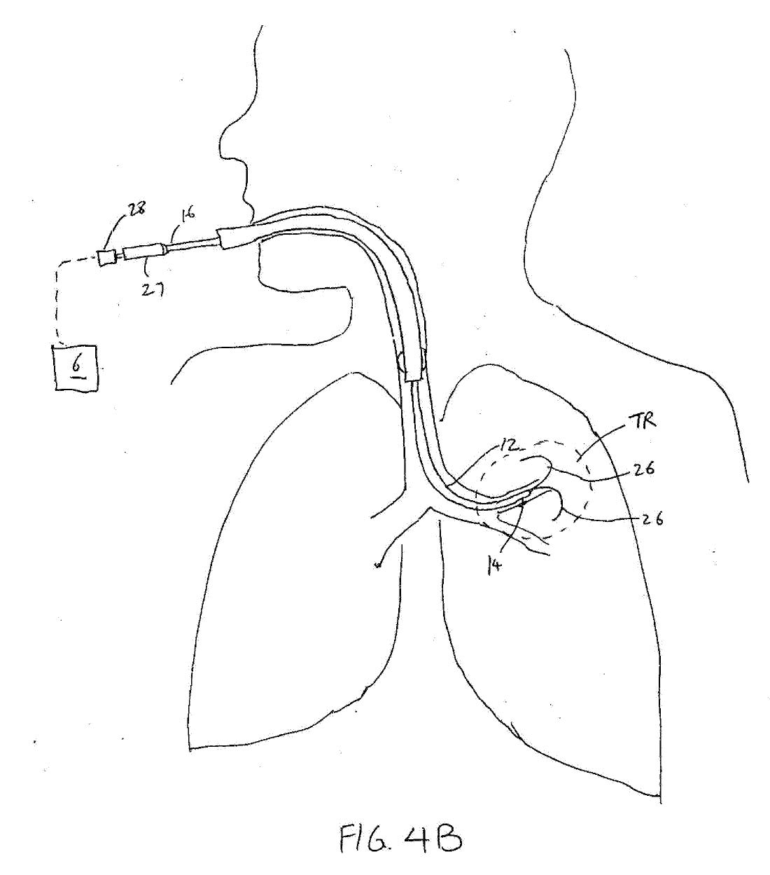

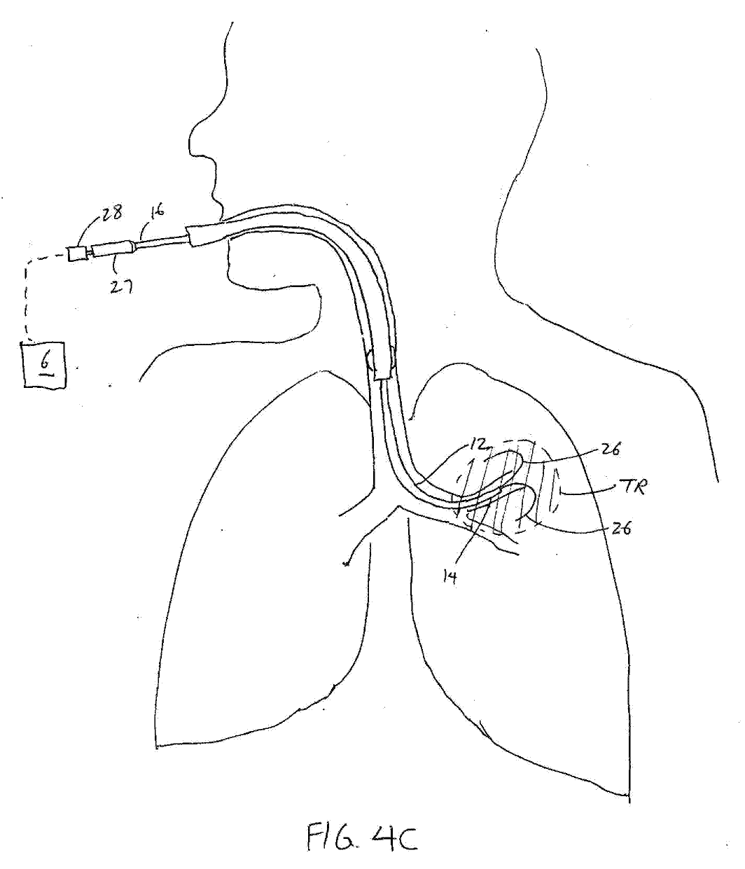

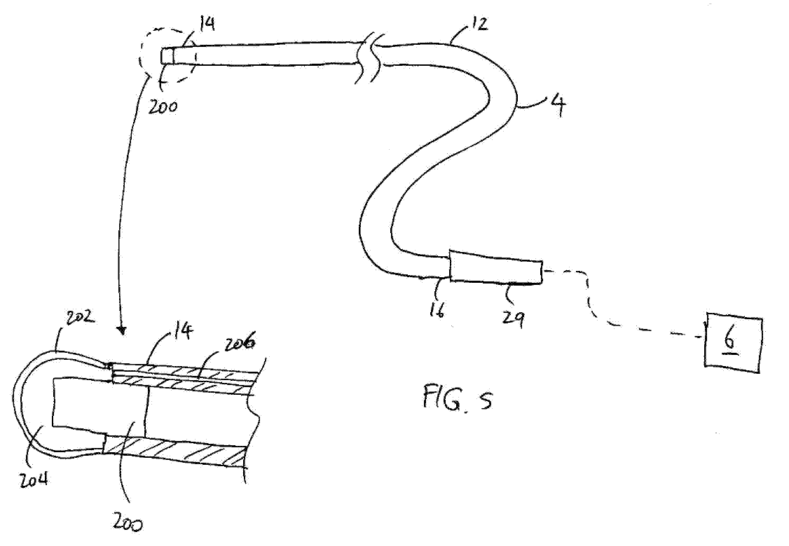

Referring now to FIGS. 4A-4C, the operation of the system 2 is described in treating a treatment region TR within lung tissue T of a patient. First, an access tube 150 is inserted into the patient's trachea (FIG. 4A). In some cases, the access tube may be implemented using a bronchoscope or an endoscope. In the illustrated embodiments, the access tube 150 may include an expandable member 152 secured to its distal end. The expandable member 152 is shown as a balloon that can be inflated using a fluid, such as gas or liquid, which is delivered to the balloon via a channel within the wall of the access tube 150. During use, the expandable member 152 is expanded to press against its surrounding (e.g., the wall of the bronchus, the trachea, or the esophagus—depending on the location of the member 152), and functions as an anchor that secures the access tube 150 relative to a patient or to another device.

Alternatively, the expandable member 152 can have other configurations. For example, in other embodiments, the expandable member 152 can be a cage that can be expanded or collapsed by manipulating one or both of a distal end and a proximal end of the cage. Expandable cage is well known in the art, and therefore, will not be described in further detail. In other embodiments, the access tube does not include the expandable member 100. In some embodiments, the treatment system 2 can further include the access tube 150. In other embodiments, instead of the expandable member 152, the system 2 may include another anchoring device. For example, in other embodiments, a bite block may be used to secure the access tube 150 at the mouth of a patient. The bite block may be made from an elastomeric/polymeric material to form a mouth insert. In such cases, the access tube 150 may have a locking component for engaging with the bite block.

Next, the cannula 12 is inserted into the access tube 150, is advanced until the distal end 14 reaches a desired location, as shown in FIG. 4A. The access tube 150 prevents the cannula 12 from contacting and sliding against the patient's esophagus as the cannula 12 is being positioned. In some cases, the access tube 150 may be tapered to allow access to increasingly smaller lumens of the lung. After the distal end 14 of the cannula 12 exits from the distal end of the access tube 150, if the cannula 12 has steering capability, the distal end 14 of the cannula 12 can be steered to the desired location (e.g., by applying tension to one or more steering wires of a steering mechanism, as is known in the art). In other embodiments, the cannula 12 may be introduced using an internal stylet or a guidewire that is subsequently exchanged for the shaft 20 and electrode array 30. In this latter case, the cannula 12 can be relatively flexible, since the initial column strength will be provided by the stylet.

After the cannula 12 is properly placed, the electrode array 30 is deployed out of the lumen 18 of the cannula 12, as shown in FIG. 4B. Particularly, the electrode array 30 is fully deployed to span at least a portion of the treatment region TR, as shown in FIG. 4B. Alternatively, the needle electrodes 26 may be only partially deployed or deployed incrementally in stages during a procedure. Next, the RF generator 6, which is connected to the treatment assembly 4 via the electrical connectors 38a 38b, is operated to deliver ablation energy to the needle electrodes 26 either in a monopolar mode or a bipolar mode. After a desired amount of ablation energy has been delivered, the targeted lung tissue at the treatment region TR collapses or reduces in volume, thereby reducing a volume of the treated region (FIG. 4C). In some cases, the reduction of the volume also causes trapped gas in alveoli (e.g., alveolar sac) of the targeted lung tissue to be removed.

If it is desired to perform further ablation to treat other lung tissue at different site(s) within the treatment region TR or elsewhere, the needle electrodes 26 may be introduced and deployed at different target site(s), and the same steps discussed previously may be repeated. When a desired amount of lung tissue at treatment region TR has been treated, the needle electrodes 26 are retracted into the lumen 18 of the cannula 12, and the treatment assembly 4 is removed from the treatment region TR.

In other embodiments, instead of accessing targeted lung tissue through the trachea and bronchus, the cannula 12 and shaft 20 may be introduced to the treatment region TR percutaneously directly through the patient's skin or through an open surgical incision. In such cases, a patient's chest is first cut opened so that at least a portion of the lung surface can be viewed by a physician. The cannula 12 is then inserted through the lung surface to reach the treatment region TR. The cannula 12 (or the electrode 92) may have a sharpened tip, e.g., in the form of a needle, to facilitate introduction to the treatment region TR. In such cases, it is desirable that the cannula 12 be sufficiently rigid, i.e., that it have an adequate column strength, so that it can be accurately advanced through lung tissue T. In other embodiments, the access of lung tissue may be performed laparoscopically using a trocar to access the inside of a body, and a scope (e.g., a laparoscope) to see the lung tissue.

After the distal end 14 of the cannula 12 has been desirably positioned, the electrodes of the ablation device 12 is then deployed into the lung, and be used to deliver energy to treat lung tissue, as similar discussed. During the procedure, a physician can determine whether the treatment region has been desirably treated by observing the surface of the lung. Since the goal of the treatment is to reduce a size of the targeted lung region, a physician can determine that the lung region has been desirably treated if the surface of the lung has subsided (e.g., due to a reduction in size of the treated tissue) during an operation.

In any of the embodiments described herein, the cannula 12 can further include a fluid delivery channel for delivering a fluid to targeted lung tissue. In some cases, the fluid delivery channel can be implemented as a lumen that is inside the wall of the cannula 12. Alternatively, the cannula 12 can carry a separate tube that provides the fluid delivery channel. During use, the fluid delivery channel can be used to deliver a conductive fluid, such as saline, to targeted lung tissue, thereby enhancing a delivery of electrical energy to targeted lung tissue.

In some cases, the delivered conductive fluid can help transmit ablation energy from the ablation electrode, and assist delivering of ablation energy to the target tissue that otherwise cannot be reached directly by the ablation electrode. In other embodiments, the fluid delivery channel can be used to deliver a toxic agent, a heated fluid (e.g., approximately 50° C. or higher), a cold fluid (e.g., approximately 3° C. or lower), or other substance that can be used to injure or scar targeted lung tissue. In still further embodiments, the fluid delivery channel can be used for delivering a needle or stylet for performing injections. For example, the needle or stylet could be carried in the channel, or otherwise be placed there through once the cannula 12 is positioned in the lung tissue.

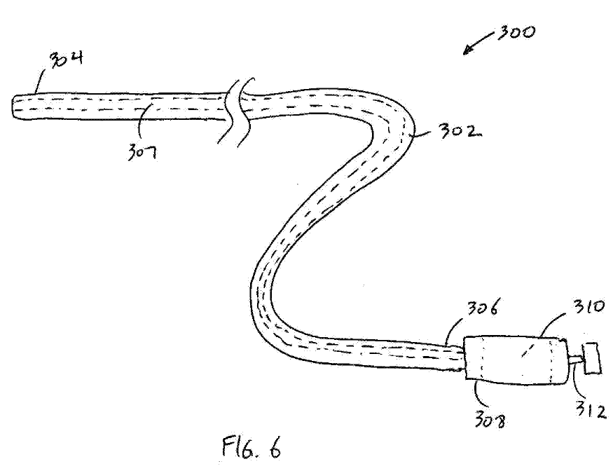

FIG. 5 illustrates a variation of the system 2 of FIG. 1 in accordance with other embodiments. The system 2 of FIG. 5 is similar to that of FIG. 1, except that the system 2 includes an ultrasound transducer 200 instead of the array 30 of electrodes 26. In the illustrated embodiments, the ultrasound transducer 200 is secured to the distal end 22 of the shaft 20. Electrical wires for driving the transducer 200 may be housed within a lumen of the shaft 20. In the illustrated embodiments, the treatment assembly 4 further includes an acoustic coupling member 202 secured to the shaft distal end 22. The acoustic member 202 has a lumen 204 that is in fluid communication with an inflation channel 206 located within the shaft 20. The inflation channel 206 is located within a wall of the shaft 20. Alternatively, a separate tube can be included for providing the inflation channel 206.

During use, the proximal end 24 of the shaft 20 is electrically coupled to the generator 6, e.g., via an electrical connector, such that the electrical wires are electrically coupled to the generator 6. The distal end 14 of the cannula 12 is then placed at a targeted treatment region. The treatment region can be accessed via the patient's trachea, or percutaneously, as similarly discussed.

After the distal end 14 of the cannula 12 has been desirably positioned, the shaft 20 is then advanced relative to the cannula 12, thereby deploying the transducer 200 out of the distal end 14 of the cannula 12. Inflation fluid, such as saline, is then delivered via the inflation channel 206 to thereby inflate the acoustic coupling member 202. The acoustic coupling member 202 expands until it presses against the wall of the bronchus (or an extension of the bronchus). The generator 6 is then activated to provide energy to the ultrasound transducer 200, thereby causing the transducer 200 to deliver acoustic energy to targeted lung tissue. As the targeted lung tissue is being treated by the acoustic energy, the lung tissue shrinks and reduces in size. As a result, any trapped gas within alveoli (e.g., alveolar sac) of the targeted lung tissue will be removed from the lung tissue.

In other embodiments, instead of placing the ultrasound transducer 200 within the lung, an ultrasound transducer can be placed adjacent to the lung surface, and be used to deliver acoustic energy to treat targeted lung tissue. For example, the ultrasound transducer can be placed or aimed in between the patient's ribs so that acoustic energy can be delivered to targeted lung tissue without being interfered by the ribs. As the lung tissue is being ablated by the acoustic energy, the tissue shrinks. In some cases, the shrinking of the tissue may remove trapped gas within alveoli (e.g., within the alveolar sac) of the lung tissue.

The treatment assembly 4 is not limited to the disclosed examples, and can include other types of ablation devices, such as a laser device that generates laser energy for ablating tissue, a heating device that generates heat for ablating tissue, a cryogenic device for delivering cooled energy (or removing heat), or some other type of device or technique known for ablating tissue. In further embodiments, the system 2 can include a source of photo-activated drug (as in a photodynamic therapy). In such cases, the treatment assembly 4 includes a light source that is configured for use with the photo-activated drug. For example, the light source can be secured to the distal end 22 of the shaft 20.

Alternatively, the light source can be secured to the distal end 14 of the cannula 12. During use, the photo-activated drug is delivered to targeted lung tissue (e.g., using the cannula 12 or another fluid delivery tube). The light source of the treatment assembly 4 is then activated to deliver light, thereby causing a photo-chemical reaction with the photo-activated drug. The reaction treats the lung tissue, and causes the lung tissue to reduce in size. The reaction may injure targeted lung tissue. As a result, the treated lung tissue shrinks and trapped gas within aveoli (e.g., alveolar sac) is removed.

FIG. 6 illustrates a system 300 for treating lung tissue in accordance with other embodiments. The system 300 includes a cannula 302 having a distal end 304, a proximal end 306, and a lumen 307 extending between the ends 304, 306. The system 300 also includes a container 308 communicatively coupled to the proximal end 306 of the cannula 302. The container 308 contains treatment fluid 310 (gas or liquid) for treating lung tissue. By means of non-limiting examples, the treatment fluid can be a drug, super-heated (e.g., approximately 50° C. or higher) or cooled (e.g., approximately ° C. or lower) liquid, or a toxic agent for killing tissue. In the illustrated embodiments, the system 300 further includes a plunger 312 coupled to the container 308. The plunger 312 is used to create a pressure within the container 308 to thereby deliver the treatment fluid 310 into the lumen 308 of the cannula 302. In some embodiments, the container 308 and the plunger 312 are implemented as a syringe.

In other embodiments, the system 300 can include one or more steering wires attached to the distal end 304 of the cannula 302. The steering wire(s) can be tensioned to thereby steer the distal 304, as similarly discussed herein.

During use, the cannula 302 is inserted into a patient's trachea, and is advanced until the distal end 14 reaches a desired location. Such can be accomplished using one of a variety of techniques. For example, an access tube, such as the access tube 150 described with reference to FIG. 4A, can first be inserted into the patient's throat. In such cases, the access tube can include an anchor balloon for anchoring against the throat of the patient, or a bite block, as discussed herein. The cannula 302 is then inserted into the access tube and exits from the distal end of the access tube to reach target tissue. In some embodiments, if the distal end of the cannula 302 is steerable, after the cannula 302 exits from the access tube distal end, the cannula 302 can be steered to reach a bronchus.

Next, the treatment fluid 310 is delivered from the container 308 to the target tissue using the cannula 302. The treatment fluid 310 causes the target tissue to shrink or reduce in size, thereby removing trapped gas within alveoli (e.g., alveolar sac) of the targeted lung tissue. In other embodiments, instead of treatment fluid 310, the container 308 may contain other substance for treating lung tissue. For example, in other embodiments, the container 308 contains radiation seed(s). During use, the radiation seed(s) is delivered within the patient's lung using a procedure that is similar to that described herein. The delivered radiation seed(s) emits radiation to treat targeted lung tissue. As the lung tissue is being treated, the tissue shrinks. In some cases, the shrinking of the tissue may remove trapped gas within alveoli of the lung tissue.

In further embodiments, the treatment fluid or radiation seed(s) can be delivered within the patient using an opened-chest procedure. In this case, after the patient's chest is cut opened, a needle can be used to penetrate the lung surface. For example, the needle can be inserted between the patient's ribs to reach the lung surface. The needle is then advanced until its distal tip reaches a desired location within the lung. Then the treatment fluid or the radiation seed(s) can be delivered into the lung using the needle. The needle is then removed from the lung. If desired, the needle can be used again to deliver additional treatment fluid or radiation seed(s) to other targeted location(s) within the lung.

In some embodiments, the seeds themselves may deliver drugs that are released into the tissue. During the procedure, a physician can determine whether the treatment region has been desirably treated by observing the surface of the lung. Since the goal of the treatment is to reduce a size of the targeted lung region, a physician can determine that the lung region has been desirably treated if the surface of the lung has subsided (e.g., due to a reduction in size of the treated tissue) during an operation.

In any of the embodiments described herein, seeds delivered into the lung can be used to conduct energy (e.g., delivered internally or externally by another device) to heat lung tissue, thereby reducing a volume of the lung tissue. In other embodiments, one or more hollow electrodes may be used to deliver the treatment fluid or seed(s) to treat lung tissue. For example, a variation of the device of FIG. 3 may be used, wherein one or more of the electrodes 26 may be hollow (e.g., have a lumen) for delivering a substance, such as the treatment fluid or seed(s). In such cases, one or more of the electrodes 26 function as needle(s) for delivering the substance.

In any of the embodiments described herein, the treatment system can further include a suction channel (not shown) for removing substance (e.g., excess treatment fluid, radiation seed, tissue, etc.) from within the patient's body. In some embodiments, the suction channel can be implemented using a separate tube (suction tube) that is located within the cannula 12 or the cannula 302. In other embodiments, the suction channel can be implemented as a channel that is embedded within a wall of the cannula 12 or the cannula 302. During use, the proximal end of the suction channel is coupled to a suction generator, which produces a vacuum for withdrawing substance into the suction channel.

Although various features of the present invention have been discussed with reference to different embodiments, it is understood by those skilled in the art that a feature of an embodiment can be combined with another feature of another embodiment of the system described herein. For example, in some embodiments, the system 2 can include a treatment assembly 4 for delivering treatment energy to treat targeted lung tissue, and also a fluid delivery channel for delivering a toxic agent. Thus, while particular embodiments have been shown and described, it should be understood that the present invention is not limited to these embodiments, and it will be obvious to those skilled in the art that various changes and modifications may be made.

For example, the array 30 of electrodes 26 can be manufactured as a single component. As such, the “array of electrodes” should not be limited to a plurality of separate electrodes, and includes a single structure (e.g., an electrode) having different conductive portions. Embodiments with multiple, i.e., axially displaced, electrode arrays are also contemplated for use with the invention, such as those disclosed in published patent applications 20040158239 and 20050080409, which are each fully incorporated by reference.

What is claimed is:

1. A system for treating lung tissue, comprising:
a fluid delivery tube having a proximal end, a distal end, and a lumen extending between the proximal and distal ends, the fluid delivery tube having a cross-sectional dimension sized for insertion into a trachea; and
a container coupled to the proximal end of the fluid delivery tube, the container in fluid communication with the fluid delivery tube, and carrying fluid for treating lung tissue;
wherein the fluid, when delivered within a lung, causes a volume of the lung tissue to reduce.

2. The system of claim 1, wherein the fluid has a temperature that is above 50° C.

3. The system of claim 1, wherein the fluid has a temperature that is below 3° C.

4. The system of claim 1, wherein the fluid comprises a toxic agent.

5. The system of claim 1, further comprising an ablation device positioned in the fluid delivery tube.

6. The system of claim 5, wherein the ablation device comprises an electrode.

7. The system of claim 5, wherein the ablation device comprises a needle configured for delivering a fluid.

8. The system of claim 5, wherein the ablation device comprises a radiation seed.

9. The system of claim 5, wherein the ablation device comprises an ultrasound transducer.

10. The system of claim 5, wherein the ablation device comprises a cryogenic device.

11. A system for treating lung tissue, comprising:
a tube having a distal end;
an anchoring device secured to the tube, the anchoring device configured to anchor at least a portion of the tube against an esophagus, a trachea, or a bronchus; and
an ablation device carried within a lumen of the tube.

12. The system of claim 11, wherein the ablation device comprises an electrode.

13. The system of claim 11, wherein the ablation device comprises a needle configured for delivering a fluid.

14. The system of claim 11, wherein the ablation device comprises a radiation seed.

15. The system of claim 11, wherein the ablation device comprises an ultrasound transducer.

16. The system of claim 11, wherein the ablation device comprises a cryogenic device.

17. A method of treating lung tissue, comprising:
inserting a distal end of a cannula into a trachea, the cannula having a distal end, a proximal end, and a lumen extending between the distal and proximal ends;
placing the distal end into a bronchus; and
delivering a fluid into a lung to reduce a volume of the lung.

18. The method of claim 17, wherein the fluid has a temperature that is above 50° C.

19. The method of claim 17, wherein the fluid has a temperature that is below 3° C.

20. The method of claim 17, wherein the fluid comprises a toxic agent.
